(19) United States
(12) Patent Application Publication
(10) Pub. No.: US 2016/0143783 A1
(43) Pub. Date: May 26, 2016

(54) Apparatus and Corneal Remodeling Methods to Improve Vision in Macular Disease

(71) Applicant: Optimal Acuity Corporation, Austin, TX (US)

(72) Inventor: Edward Allen Yavitz, Rockford, IL (US)

(21) Appl. No.: 14/604,850

(22) Filed: Jan. 26, 2015

Related U.S. Application Data
(60) Provisional application No. 62/083,656, filed on Nov. 24, 2014.

(51) Int. Cl. A61F 9/008; A61B 18/08; A61B 18/14

(52) U.S. Cl. CPC A61F 9/00827; A61B 18/14; A61F 9/00834; A61F 9/0084; A61B 18/082; A61B 2018/144; A61B 2018/00994

(57) ABSTRACT

Apparatus and methods to improve the vision in a person with a macular disease, comprising the purposeful temporary creation of increased corneal optical aberrations. Strategies include corneal treatment patterns that are one or more of asymmetric, decentered or eccentric with respect to the visual axis, which would result in worsening vision in a normal eye but surprisingly become sight enhancing in a patient with the loss of foveal function due to macular disease.

### CROSS REFERENCE TO RELATED APPLICATION

The present application claims priority to co-pending U.S. provisional patent application No. 62/083,656 filed Nov. 24, 2014, and entitled “APPARATUS AND CORNEAL REMODELING METHOD TO IMPROVE VISION IN MACULAR DISEASE”, the entire contents of which is incorporated by reference herein.

### FIELD OF THE INVENTION

The present invention relates to apparatus and laser means to alter the shape of the human cornea in order to improve the vision in a person with macular disease.

### BACKGROUND OF THE INVENTION

The retina is the light sensitive portion at the back of the human eye which receives light rays focused by the cornea and lens at the front of the eye. In a normal eye (FIG. 1) light is reflected from an object (2) and is focused by the cornea (3) and lens (4) to the 0.5 mm diameter circular fovea (5) of the retina, the area of maximal visual acuity. The fovea is at the center of a 5 mm diameter circular retinal area called the macula (6). The visual axis (1) is defined as a straight line extending from the viewed object (2) through the center of the cornea and pupil (7) to the fovea (5). Color and sharp visual acuity are at their peak in the fovea and as the distance increases from the fovea, the number of photoreceptors drops, lowering the potential acuity. One degree of displacement from the fovea is ca. 0.3 mm and five degrees displacement corresponds to ca. 1.5 mm displacement. In a normal eye the fovea has a resolution of potentially better than 20/20, but the potential vision 1.5 mm away from the fovea is only 20/70, the legal limit for driving a car. In an abnormal eye with retinal macular disease, commonly known as AMD, there is a loss of photoreceptors in the fovea and surrounding macula with usually little or no potential acuity in the fovea and for several degrees from the center of the fovea. The remaining functioning retina can potentially yield 20/200 to 20/400 vision. AMD causes progressive loss of central vision and therefore loss of the ability to function as one would desire in society for 15 million Americans. There is no known cause or effective treatment for the dry type of AMD. Millions of other people have related debilitating macular disorders such as chronic macular edema, macular pucker, Stargardt's Disease, Best's Disease, macular holes and Geographic Atrophy. It would be highly advantageous to have an apparatus and method to improve the vision and ability to function for these people. The present invention does that by taking advantage of a feature that all of these diseases have in common. Despite the fact that the fovea may be destroyed and non-functioning, there are usually small areas of the macula near the fovea that still have viable photoreceptors. These islands of functioning retina are called preferred retinal locations (PRLs). Early in most macular diseases, there are PRLs very close to the fovea which can potentially produce vision up to 20/50 by Snellen chart measurements but not usually up to the level of 20/20 as in the fovea. Later as the diseases progress, even these close PRLs are destroyed and only more distant PRLs are still functioning. Since the ability of the macula to resolve light into sight is greatest in the fovea and drops off rapidly with the distance from the fovea, the potential vision decreases as the distance of the PRLs from the fovea increases.

The advent of a device called a micro perimeter which combines eye tracking with visual field testing in the macula region demonstrates the PRLs present in these diseases. FIGS. 2 and 3 are screen shots from a microperimeter study of representative forms of macular disease. FIG. 2 is Geographic Atrophy and FIG. 3 is Best's disease. The macular regions (6) bounded by blood vessels (22) from the optic nerve (21) have lost their photoreceptors and appear white. The PRLs (24) in FIGS. 2 and 3 found by the microperimeter are shown as dark dots (24). There can be one or more PRLs which represent still functioning areas of retina near the diseased fovea (5) and macula (6).

Prior to the present invention, patients with macular degeneration could sometimes improve their vision by moving an image onto PRL areas of the macula outside of the fovea using prisms in their spectacles or with telescopes mounted on their spectacles or hand held magnifiers for close work. A prism shifts the image location but causes double vision when both eyes are open. For this reason forming a prism out of the corneal tissue with ablation techniques or implanting a prism inside the cornea, methods taught by Macoul et al. in U.S. Pat. No. 5,984,961 and Azar in U.S. Pat. No. 5,634,919 have failed to live up to their theoretical ability to move the image to a retinal location outside the fovea. In practice, their techniques create double vision and move the image on a permanent basis to an area of the macula that will soon becomes non-functioning due to the progression of disease. In a group of diseases that are progressive, it is essential to the success of any treatment that it be temporary so that as available PRLs change, it can be repeated. Furthermore, patients often have multiple PRLs in different axes as shown in FIG. 3. Since a prism can only move the image in one axis, PRLs in all other axes are missed. The present invention overcomes these limitations.

Telescopes enlarge the image size so that its footprint upon the retina is enlarged to include all PRLs close to the fovea. Unfortunately, the concomitant loss of peripheral vision with telescopes make such methods problematic. Recently, telescopic intraocular implants such as those taught by Azar et al. in U.S. Pat. No. 8,506,626, have been placed into corneas or used to replace cataracts in patients with macular disease according to Peyman in U.S. Pat. No. 7,186,266 and Portnoy in U.S. Pat. No. 4,759,761. Again, severe loss of peripheral vision and extreme cost and complexity make them impractical. The present invention temporarily enlarges and moves an observed image to multiple PRLs without reducing peripheral vision and without the use of prisms or telescopes.

The eye, like any other optical system, suffers from a number of optical aberrations which reduce vision. Correction of spherocylindrical refractive errors, namely myopia, hyperopia and astigmatism has been possible for nearly two centuries following Airy's development of methods to measure ocular astigmatism. These so-called lower order aberrations have been corrected with spectacles, contact lenses, intraocular lenses and refractive corneal surgery such as radial keratotomy and laser corneal reshaping surgery such as LASIK. None of these techniques effectively correct the higher order aberrations of spherical aberration, coma and trefoil which fortunately do not often occur naturally.

At the risk of putting the reader into a coma a brief discussion of higher order aberrations including coma is necessary. Light coming off of a perceived object can be described either as discrete rays or as a wave of light perpendicular to those rays.

For light to converge and focus to a perfect point such as the fovea of the eye, the wavefront emerging from the optical system comprising the cornea and lens of the eye must be a perfect sphere centered on the fovea. This is seen in FIG. 4 using what is called Zernike polynomial calculations. The distance in micrometers between the actual wavefront and the ideal spherical wavefront is called the wavefront aberration, which is the standard method of describing the optical aberrations of an eye. Aberrations of the eye are the difference between two surfaces: the ideal and the actual wavefront.

Ophthalmologists, optometrists and opticians exist because no eye is ideal. In the normal population the most prevalent optical aberrations are second-order spherocylindrical focusing errors, which are called refractive errors such as myopia (nearsighted), hyperopia (farsighted) and astigmatism. In myopia, distant objects are defocused while near objects are in focus at the fovea. In hyperopia both distance and near objects are out of focus as their wavefronts strike the retina. Both myopia and hyperopia result in defocus, seen in FIG. 4 as Z₂⁰. In astigmatism, objects appear doubled, usually vertically or horizontally because while most of their light rays hit the fovea, some of them simultaneously strike areas outside of the fovea observed as a second shadow image, and shown in FIG. 4 as Z₂² and Z₂⁻². Of importance to the present invention, in patients with the loss of foveal function, instead of seeing a double image when corneal astigmatism is present, only the secondary image striking a PRL is visible, while the primary image striking the fovea is not seen.

Higher order aberrations are a relatively small component of typical visual disturbances, comprising about 10% of the normal eye's total aberrations and include spherical aberration, coma and trefoil. Spherical aberration results in observed halos around point images and is treated with pupil constricting drops. In optics (especially telescopes), the coma, or comatic aberration, refers to an aberration due to imperfection in a lens that results in point sources such as stars appearing enlarged, having a comet-like tail, hence the term coma. In fact, coma is often defined as an image enlargement. Coma is portrayed in FIG. 4 as Z₃⁻¹ and Z₃¹, and would be seen in a normal eye as an enlarged image, striking both the fovea and areas outside the fovea. The present inventor has discovered this normally annoying feature to be useful in those with loss of foveal function but with functioning PRLs that can interpret the enlarged image. The last higher order aberration of consequence, trefoil, causes multiple images to appear in the periphery of vision and is shown in FIG. 4 as Z₃³ and Z₃⁻³. When corneal trefoil is present, the primary image hits the fovea but three additional images are projected at 1two0 degree intervals outside the fovea. Finally, the present invention can induce the optical aberation of tilt shown in FIG. 4 as Z₁⁻¹, Z₁¹ which will shift the image outside the fovea in one direction, for example to the right or below the fovea.

While there is copious prior art covering the use of lasers and intracorneal inlays to change the shape of the human cornea, it mainly concerns itself with improving the refracting ability of the normal eye so that light from a perceived object can be best focused upon the fovea of the eye. Such prior art teaches methods to reduce pre-existing optical aberrations of the eye such as myopia, hyperopia and astigmatism while limiting any aberrations that might be caused by the treatment. Most LASIK procedures remove collagen from the central cornea to reduce the corneal sphericity, reducing its refractive power and improving myopia. Customized LASIK wavefront-guided refractive corneal laser treatments are designed to reduce existing aberrations and to help prevent the creation of new aberrations. Alignment of the treatment and the pupil is usually achieved through iris feature detection and eye tracking so that the treatment is perfectly centered around the patient's pupil. In another technique called thermal keratoplasty, a laser is used to heat-shrink collagen in peripheral areas of the cornea in a symmetric pattern centered on the pupil in order to cause the center of the cornea to bulge outward, making the cornea more convex and prolate to decrease hyperopia or presbyopia. If one skilled in the art were to follow each of the exemplary techniques and methods of the prior art in this field, they would not be able to improve the vision in a person with a macular disease which has destroyed foveal function. In fact, all operative guidelines as well as the labeling guidelines of the FDA forbid the use of such lasers and techniques in patients with retinal disease as being contraindicated (if not pointless).

U.S. Pat. No. 8,852,176 issued to Riedel et al. and U.S. Pat. No. 8,764,737 to Kurtz et al. describe image-guided methods to insure that a refractive laser will deliver a treatment that is well-centered upon the pupil.

U.S. Pat. No. 8,663,208 awarded to Bor teaches intrastromal laser refractive correction comprising a plurality of intrastromal incisions radially oriented about an optical axis of the eye.

U.S. Pat. Nos. 8,617,146 and 8,409,179 issued to Naranjo-Tackman et al. and to Bille et al. respectively describe a system, apparatus and methods for laser cataract surgery as well as making cuts in the corneal periphery centered on the pupil for the purpose of reducing astigmatism.

U.S. Pat. Nos. 8,556,886 and 5,891,132 to Hohla teaches an iris recognition system and an earlier topography system that measures abnormalities in the patient's corneal surface topography and translates that into a laser treatment pattern to counteract and correct the refractive error caused by those abnormalities. He describes the possibility of non-standard treatment, using manually or semi-manually placed shots but only in order to counteract and correct for pre-existing irregular corneal abnormalities such as hot spots, curved and irregular astigmatism in order to bring light to focus on the fovea.

There have been a few other non-standard or eccentric patterns of excimer laser corneal ablations proposed in the prior art. U.S. Pat. No. 8,529,558 to Stevens describes a laser ablation method to correct pre-existing or post-refractive surgery higher order optical aberrations and to better focus light on the fovea of the eye. Clapham in U.S. Pat. Nos. 6,245,059, 6,572,607 and 7,004,935 also teaches the purposeful off-center ablation of an irregularly shaped cornea with an excimer laser for the purpose of making the cornea more spherical and reducing higher order optical aberrations. The goal in each of the foregoing patents is to improve the cornea's curvature to better focus light onto the fovea, even if that requires non-standard patterns of corneal ablation. Another non-standard pattern is proposed by Kuo in U.S. Pat. No. 8,298,214 in which an excimer laser unit is used to form multiple convex arcs on a cornea of an eyeball of a person so that the healthy person or the person with refractive errors can also prevent oncoming presbyopia by virtue of having multiple defocused images on the fovea that one day might be useful as presbyopia progresses. The only prior art that contemplates a non-standard eccentric pattern of excimer corneal ablation in order to shift at least part of the image onto still functioning areas of the macula that are not affected by macular degeneration is in U.S. Pat. Nos. 8,192,023, 7,871,163 and 7,874,672 awarded to Grierson and Lieberman. In their teaching, an excimer laser is used to permanently create multiple arcs as in the Kuo patent above but altering their position relative to the pupil such that the cornea is reshaped to refract the light from a perceived object into a plurality of points of focus so as to form a predetermined pattern, one of a circle, a spiral, a rose pattern and a dual rose pattern upon the retina. By decentering the multiple ablation arcs, Grierson and Lieberman create a focused image at a distance of only up to 0.01 mm from the fovea. The present invention overcomes two problems with the Grierson and Lieberman art. First, the prior art alters the corneal shape permanently by removing corneal tissue with excimer laser ablation, when in fact macular diseases are progressive, wherein available locations are likely to change over time. The apparatus and methods described by the present invention create temporary, alterable changes to the corneal shape. Secondly, the prior art taught in the Grierson and Lieberman patents can only decenter a focused image up to 0.01 mm from the fovea whereas the present invention moves the image by several hundred fold up to 3 mm from the fovea where many PRLs still exist in patients with advanced macular disease.

U.S. Pat. No. 8,454,167 given to Seiler, et al. outlines a thermal laser that corrects presbyopia by shrinking corneal collagen symmetrically around the visual axis, increasing the convexity of the central cornea.

U.S. Pat. No. 8,444,632 to Reinstein et al. emphasizes the importance of centering refractive laser eye surgery along the visual axis of a human eye.

U.S. Pat. No. 8,409,177 to Lai and incorporated herein by reference teaches a method to inducing a corneal shape change for treating a keratoconus condition, or correcting a refractive error or a high order aberration, or combinations thereof, and particularly to generating an intrastromal pocket within a patient's eye with a laser and filling the pocket with a polymeric insert. Other similar patents incorporated herein by reference include Zickler et al. in U.S. Pat. No. 8,246,609 in which a corneal pocket is filled with human donor cornea and U.S. Pat. No. 6,551,307 by Peyman, who teaches the use of a biocompatible gel to fill a corneal pocket to modify the cornea to correct refractive error.

U.S. Pat. No. 8,052,674 to Steinert et al. describes one of many methods to track eye movement in order to avoid decentering the treatment. U.S. Pat. No. 6,626,896 to Frey et al. is another example of eye tracking to keep the laser treatment centered on the visual axis so that after surgery, light from an image will focus on the fovea.

U.S. Pat. No. 6,530,917 granted to Seiler et al. describes a device and method to measure the wavefront optical aberrations of the entire optical system of the eye and creates an ablation profile, calculating the layer thickness of the corneal collagen to be permanently removed at various sites in order to reduce or eliminate higher order aberrations and to better focus light on the fovea.

U.S. Pat. No. 6,344,039 to Targ et al. describes a microscope viewing system to reduce parallax errors which can inadvertently cause a decentered laser ablation.

It was only through a surgical error in a Phase I FDA blind eye study of a thermal laser that the present invention was discovered. An inadvertent and totally decentered treatment with a thermal laser in a patient with late stage AMD surprisingly improved the patient's vision to a remarkable degree. Understanding how that happened is the basis for the present invention.

### SUMMARY OF THE INVENTION

After careful screening for suitable candidates with macular disease, in one method, a femtosecond laser is used to create an off center pocket within the cornea for the placement of a spacing material such as hydrogel contact lens or human donor cornea material and in another method a thermal laser is used to produce a pattern of corneal collagen shrinkage that is one or more of asymmetric, decentered or eccentric with with respect to the visual axis and in a third method, a thermal conducting wire is used in place of a thermal laser to create collagen shrinkage in a similar pattern with respect to the visual axis. These methods temporarily create a more convex altered corneal topography to purposely increase one or more of the optical aberrations of myopic defocus, astigmatism, coma, trefoil, tilt and tetrafoil which moves light rays from an observed object to preferred retinal locations away from a diseased fovea. This stimulates the photoreceptors in PRLs comprising functional non-foveal areas of retina located 0.01 to 3.0 mm from the diseased fovea to improve the vision in persons with macular disease. All of these techniques are meant to be temporary, lasting from several months to several years so that as the retinal disease progresses, additional treatments can be performed to place the observed image onto remaining PRLs more distant from the fovea. In the case of corneal pockets, the material can be easily removed after which the pocket naturally heals and another pocket in a more eccentric location can be made. In the case of the thermal laser, a wavelength and technique are chosen that produce a temporary change to the cornea and can be repeated in the same or different locations after the cornea returns to its original shape.

It is to be understood that both the foregoing general description and the following detailed description are exemplary and explanatory only, and are not restrictive of the invention.

### BRIEF DESCRIPTION OF THE DRAWINGS

Non-limiting and non-exhaustive embodiments of the present invention are described with reference to the following. FIGURES, wherein like reference numerals refer to like parts throughout the various views unless otherwise specified.

FIG. 1 depicts a schematic drawing of a normal eye showing pertinent anatomical features and the pathway of light from an observed object to the retina.

FIG. 2 depicts a screenshot of a microperimetry report of the macula of a patient with the macular disease Geographic Atrophy.

FIG. 3 depicts a screenshot of a microperimetry report of the macula of a patient with the macular disorder known as Best's disease.

FIG. 4 depicts images of Zernike polynomials representing the most common optical aberrations found in the visual system.

FIG. 5 depicts a drawing of an eye which has had a corneal pocket created by a femtosecond laser and filled with a clear contour changing material which in turn changes the pathway of light from an observed object to the retina when compared to the normal schematic eye of FIG. 1.

FIG. 6a depicts the anatomical landmarks of an untreated corneal surface.

FIG. 6b depicts the resulting projection of light rays from an observed object through the cornea of FIG. 6a onto the retina.

FIG. 6c depicts the thermal treatment locations of a cornea treated with a thulium laser.

FIG. 6d depicts the resulting projection of light rays from an observed object through the treated cornea of FIG. 6c onto the retina.

FIG. 6e depicts the thermal treatment locations of a cornea treated with a thulium laser.

FIG. 6f depicts the resulting projection of light rays from an observed object through the treated cornea of FIG. 6e onto the retina.

FIG. 7 depicts a screenshot of the pre and post operative ray tracing retinal spot diagram from an iTrace examination of the patient in Example 1.

FIG. 8 depicts a screenshot of the pre and post operative ray tracing retinal spot diagram from an iTrace examination of the patient in Example 2.

FIG. 9 depicts a screenshot of the pre and post operative ray tracing retinal spot diagram from an iTrace examination of the patient in Example 3.

### DETAILED DESCRIPTION OF THE INVENTION

The present invention first requires selection of patients who have a cornea without pre-existing irregular astigmatism, scars or corneal dystrophies and who have lost all or most of their foveal function but still have islands of functioning parafoveal photoreceptors, termed PRLs. In addition, the patient should not have a dense cataract. This selection process requires a standard slit lamp examination of the cornea and lens of the eye as well as photographic and indirect ophthalmoscopic examination of the retina. A phoropter refraction using an American Optical phoropter for example as well as auto refraction and topography with for example a NIDEK OPD II machine from NIDEK Corporation, Japan and a ray tracing using the iTrace machine made by Tracey Corporation of Houston, Tex. will rule out patients with irregular astigmatism, corneal scars or dystrophies and cataracts who are not good candidates for this procedure. A microperimeter made by Centervue Inc of San Jose, Calif. is then used to confirm loss of central foveal function and to locate PRLs of still functioning macular regions.

In one exemplary method shown schematically in FIG. 5, a femtosecond laser such as the VICTUS made by Bausch and Lomb/Technolas is used to create a corneal pocket (11) inside of the cornea (3). The corneal pocket is then filled with a clear contour-changing material (12) such as a hydrogel contact lens or a donor corneal lenticle. The pocket is purposely decentered from the corneal center (14) in order to induce tilt, coma and astigmatism. The light rays (1) from a viewed object (2) no longer focus upon the fovea (5) as in the normal schematic eye of FIG. 1, but now are skewed to a new retinal location (13) apart from the fovea (5), where a functioning PRL exists. Later, if the macular disease progresses, requiring increased displacement of the image to remaining PRLs more distant from the fovea, this technique can be repeated by removing the inserted material, allowing the corneal pocket to heal for a few months and making a new pocket more eccentric to the visual axis in order to create even more astigmatism and coma in order to move the light rays from the image even farther from the fovea to the functioning PRLs.

In another exemplary method, a thermal laser such as the thulium laser (Optimal Acuity Corporation, Austin, Tx) is used to remodel the cornea, making topographic and contour changes to produce a pattern of increased corneal convexity which is one or more of asymmetric, decentered, or eccentric in relationship to the visual axis of the cornea in order to purposely increase optical aberrations which moves the reflected light from an observed object to still functioning PRL areas up to 3 mm from a diseased fovea. The more asymmetric, decentered and/or eccentric that the treatment pattern is, the farther away from the fovea the light from the object will be projected. However, since the potential resolution of the retina decreases rapidly with distance from the fovea, it is important to keep the image as close to the fovea as possible provided that there are PRLs available. While this is likely early in a macular disease, as the macular disease progresses, PRLs close to the fovea may be lost, leaving only more distant PRLs with lower potential visual resolution available. Since the treatment of the present invention is designed to be temporary, the cornea will gradually return to its original shape and allow the physician to repeat the procedure with a more eccentric pattern in order to reach the remaining PRLs later in the disease.

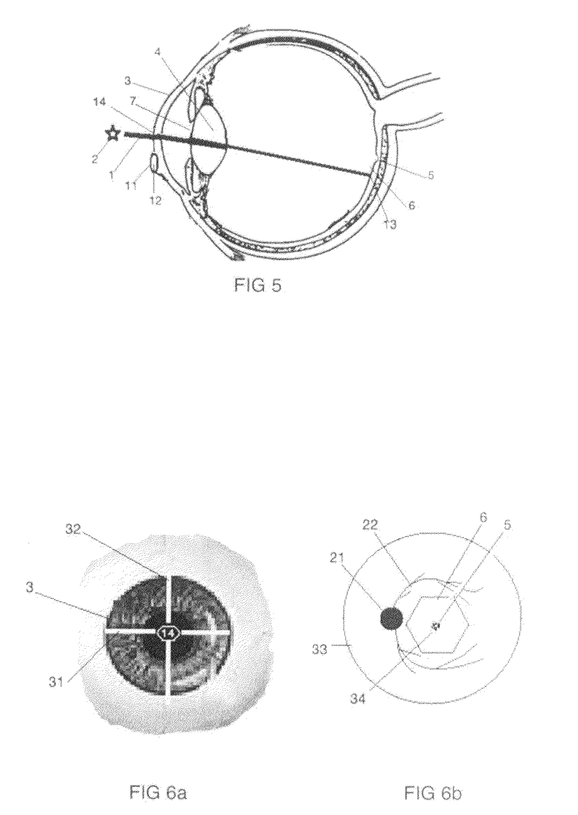

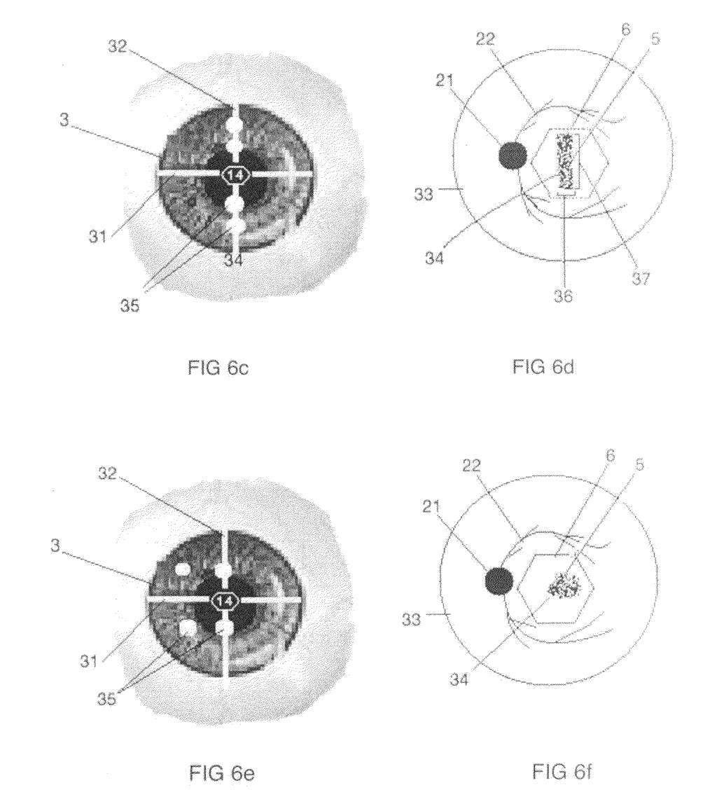

Possible strategies for patterns of thermal treatment on the cornea of a patient with macular disease and their resulting light ray patterns on the retina are shown in FIG. 6 a-f. The thermal laser treatments on the cornea are made eccentric, asymmetric or both with respect to the corneal and pupillary center to produce optical aberrations.

The thulium laser thermal treatment pattern on the cornea surface is shown in FIG. 6 a, c, e and the resulting projection of the light rays on the retina in FIG. 6 b, d, f. FIG. 6a shows an untreated cornea (3) with a corneal x axis (31), a y axis (32) and a corneal center (14), usually coinciding with the pupillary center. The light rays from an observed image will be focused as shown in FIG. 6b to the retina (33), in a compact pattern (34) on the fovea (5) at the center of the macula (6) which is seen bounded by retinal vessels (22).

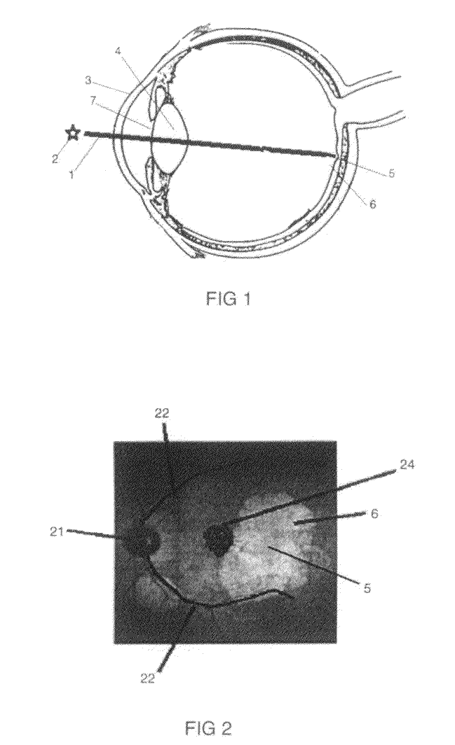

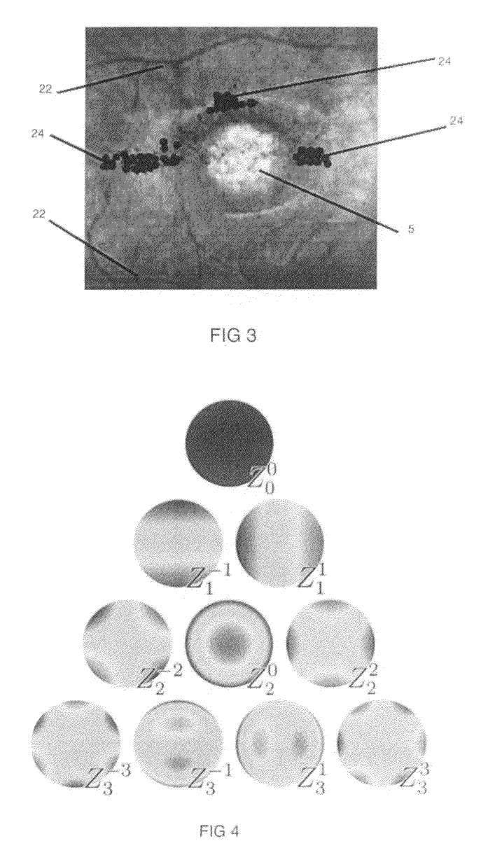

FIG. 6b shows a purely asymmetric treatment of thermal spots (35) only in the y axis (32) but centered on the corneal center (14) which causes an increase in corneal convexity along the y axis and creates the image pattern (34) on the retina shown in FIG. 6d which is thicker (36) and more spread out (37) along the y axis than the pattern (34) in FIG. 6b due to newly created myopic defocus and astigmatism. This is desirable when PRLs are found above and/or below the fovea on preoperative microperimetry. in FIG. 6e where the treatment spots (35) are to the left of the corneal center (14) and the resulting image pattern (34) shown in FIG. 6f is displaced to the right of the fovea (5). The more eccentric to the visual axis this asymmetric treatment pattern is, the greater the shift of the image. This is desirable when there are PRLs found to one side of the fovea on pre-operative mlcroperimetry studies as shown in FIG. 2. A treatment spot can be placed directly on the corneal center in a patient with poor macular function without hurting the vision. In a normal eye, this would result in loss of vision.

The following examples are given to illustrate the scope of the present invention. Because these examples are for illustrative purposes only, the invention should not be inferred to be limited to these examples. Other methods besides those in the examples and those already described herein and known to those skilled in the art for altering the topography of the cornea including the use of wires conducting heat or radio frequency waves, ultrasonic waves, and the use of other lasers such as holmium or even excimer in extreme cases could be used according to the present invention to remodel and alter the topography of the human cornea and thereby purposely create optical aberrations such as tilt, coma, astigmatism and trefoil in order to move the light from an observed object to still functioning PRL areas away from a diseased fovea. Other optical aberrations could be created in addition to astigmatism and coma for this purpose such as myopic defocus, trefoil, prism, and tetra foil. The treatment of the present invention could be beneficial in a large number of retinal disorders including but not limited to macular degeneration, geographic atrophy, Best's disease, Stargardt's disease, chronic macular edema, macular hole, macular pucker, chloroquine maculopathy, solar maculopathy, angloid streaks, cone degenerations, choroidal folds, chronic epiretinal membrane, chronic central serous maculopathy, and foveal loss due to histoplasmosis, toxoplasmosis, or amoebiasis. The laser and method used in the following examples produces a temporary alteration in the shape of the cornea, which will gradually return to its original shape so that the cornea can be retreated at a later time using a more eccentric pattern if the macular disease progresses and the distance of remaining PRLs from the fovea increase; however, in extreme cases where the disease is at its full extent and PRLs are at a maximal distance from the fovea to allow for any useful visual resolution, a final and permanent treatment eccentrically removing corneal tissue with an excimer or femtosecond laser might be necessary. In such patients, the temporary effect of a thermal laser or method could also be made permanent through the use of a technique known in the art as riboflavin cross linking.

### Example 1

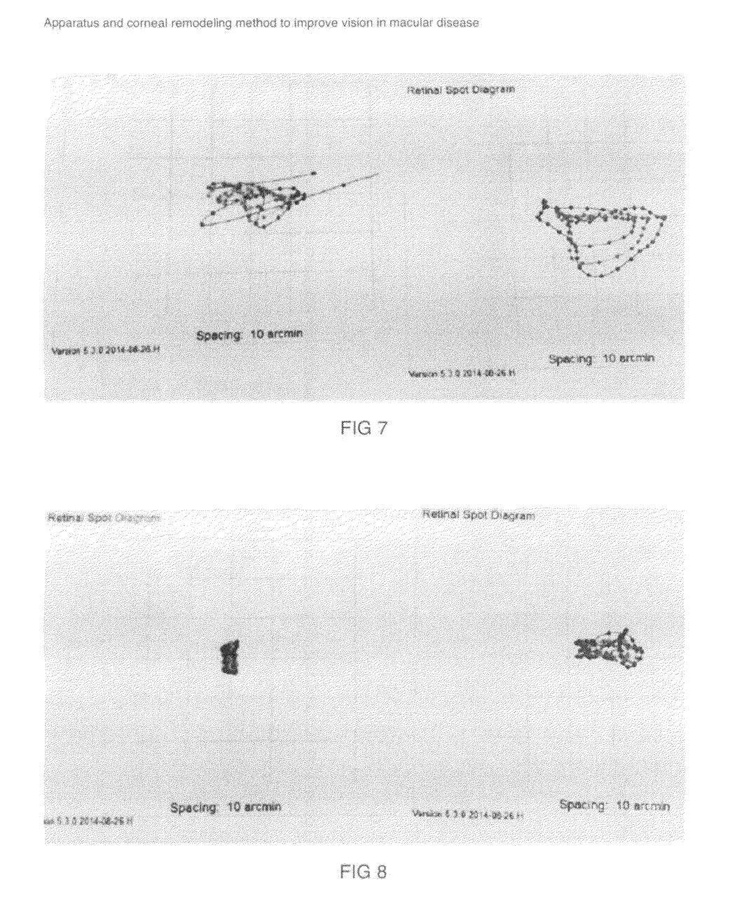

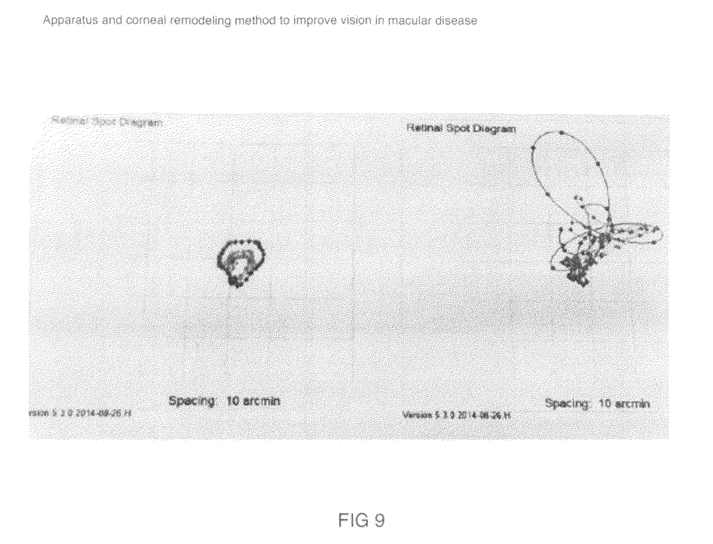

A patient with macular degeneration and best corrected vision of 20/400 in the right eye with no reading vision (less than Jaeger 10 meaning he could not read even the largest print) underwent a four spot treatment with a thulium laser at a power of 46 millijoules per spot. The four spots were equidistant from each other at the corners of a square 6 mm×6 mm in size. Rather than centering the square with the pupil in the center, it was decentered laterally on the cornea by 4 mm as shown in FIG. 6c. The iTrace showing the pre and post operative retinal spot diagram shows the ray tracing pattern of light from the image upon the retina is seen in FIG. 7. The grid in the figure is 1 mm×1 mm of the patient's retina centered on the fovea. Each box represents 10 arc minutes or 0.16 mm of the retina. It should be noted that the typical fovea is only 0.3 mm in diameter so that any ray tracings outside of the central 4 boxes is outside the fovea where PRLs may exist. The preoperative tracing of the image upon the retina on the left side of FIG. 7 is 0.16 mm×0.32 mm in size and covers the entire fovea area. The computer of the iTrace machine (Tracey Technology) which took these tracings also calculated the total optical aberrations preoperatively to be 0.45 microns, with astigmatism causing 0.2 microns, coma 0.03 microns and trefoil 0.05 microns, and the remainder being myopic defocus. These calculations are not shown in the FIGURES, but are taken directly from the iTrace readouts. Post operatively the post-op image shown on the right side of FIG. 7 is obviously larger, subtending 0.3 mm×0.5 mm and extends beyond the fovea. It also has shifted below and to the right of the fovea. FIG. 7 shows the change in the image size and location upon the patient's retina after laser treatment. The total optical aberrations were measured by the iTrace to have increased from 0.45 to 0.65 microns, with astigmatism rising from 0.20 preoperatively to 0.35 microns and coma increasing from 0.03 microns to 0.08 microns. Trefoil doubled from 0.05 to 0.10 microns. Most importantly, the patient had improved vision. His best corrected distance vision improved from 20/400 to 20/80 and his reading improved from less than Jaeger 10 to Jaeger 6.

### Example 2

A female patient with early AMD had best corrected vision of 20/200 but could read only Jaeger 10 size print. She underwent the exact same procedure as in Example 1. Her ray tracings are shown in FIG. 8. Her preoperative image on the left side of FIG. 8 subtended only 0.1 mm×0.16 mm precisely on her fovea. It appears smaller than the preoperative image of Example 1 because he had preoperative myopia and optical aberrations of 0.45 microns which spread the image out. Her preoperative total optical aberrations were only 0.12 microns which included astigmatism of 0.1 microns, coma of 0.01 microns and trefoil of 0.01 microns and no contribution from myopia or hyperopia. Post operatively, her ray tracing on the right of FIG. 8 had expanded to 0.3 mm×0.3 mm and had spread both vertically and horizontally to just beyond the edge of the fovea, where the potential acuity is almost as good as in the fovea. She did indeed achieve her potential acuity, improving to 20/50 vision and Jaeger 6 reading ability. Her post operative total optical aberrations had increased to 0.45 microns from the 0.12 microns preoperatively. This was due to an increase in astigmatism to 0.30 microns, coma to 0.07 microns and trefoil to 0.08 microns.

### Example 3

In this woman with geographic atrophy of the macula which had destroyed her fovea and much of the macula, only a PRL more than 0.5 mm above the fovea could be detected by preoperative microperimetry (Centerview Inc, San Jose, Calif.). In order to spread the image that far above the fovea, a 4 spot pattern with the thulium laser using 48 mJ of power was decentered such that one spot was directly on the visual axis, upon the pupil center as shown in FIG. 6d. The preoperative best corrected vision was 20/800 and near vision was less than Jaeger 10. The post operative vision improved to 20/200 and she could read Jaeger 8 size print. The ray tracing in FIG. 9 explains this dramatic improvement. Pre operatively the image subtended 0.2 mm in the fovea of the retina as seen on the left side of FIG. 9, while post operatively it had spread to 0.6 mm×0.8 mm in size, with the image hitting her PRL between 0.5 and 0.6 mm above her fovea. This was accomplished by increasing her total optical aberrations from 0.2 microns to 0.6 microns and tripling her astigmatism from 0.15 to 0.45 microns, her coma from 0.10 microns to 0.4 microns and increasing her trefoil fifty-fold from 0.006 to 0.28 microns. The distinctive three petal shape of trefoil in the post-op retinal spot diagram is dramatic and accounts for the image being expanded well beyond her fovea by 0.8 mm, enough to hit her PRL 0.5 mm from her fovea and allow for improved vision.

I claim:

1. An apparatus for temporarily altering the topography of the human cornea to purposely increase optical aberrations in order to move light rays from an observed object to preferred retinal locations away from a diseased fovea.

2. The apparatus of claim 1 comprising one of a hydrogel contact lens, an ultrasound, a laser, a thermal conducting wire or a radio frequency conducting wire.

3. The apparatus of claim 1 comprising one of a thulium laser, an excimer laser, a holmium laser and a femtosecond laser.

4. The alteration of corneal surface topography of claim 1 comprising increased convexity which is one or more of asymmetric, decentered, or eccentric with respect to the visual axis.

5. The optical aberrations of claim 1 comprising one or more of defocus, prism, tilt, astigmatism, coma, trefoil and tetrafoil.

6. A diseased macula of claim 1 comprising one of macular degeneration, geographic atrophy, Best's disease, Stargardt's disease, chronic macular edema, macular hole, macular pucker, chloroquine maculopathy, solar maculopathy, angioid streaks, cone degenerations, choroidal folds, chronic epiretinal membrane, chronic central serous maculopathy, foveal loss due to histoplasmosis, toxoplasmosis, amoebiasis.

7. The preferred retinal locations of claim 1 comprising non-foveal areas of retina located 0.01 to 3.0 mm from the diseased fovea which show functional sensitivity on a preoperative microperimetry examination.

8. A method of treatment of a diseased macula comprising temporary alteration of the surface topography of the human cornea to purposely increase optical aberrations in order to move the light rays from an observed object to functioning preferred retinal locations away from the diseased fovea.

9. The method of alteration of the surface topography of the human cornea of claim 8 comprising creation of a decentered pocket in the cornea using a femtosecond laser and filing the pocket with one or more of a donor corneal lenticle or a hydrogel contact lens material.

10. The method of treatment claim 8 comprising creation of a decentered pattern of corneal collagen alteration with one or more of a femtosecond laser, excimer laser, thulium laser, a holmium laser, an ultrasound, a thermal conducting wire or a radio frequency conducting wire.

11. The treatment of claim 8 which includes a treatment directly on the visual axis.

12. The alteration of surface topography of the human cornea of claim 8 comprising increasing convexity which is one or more of asymmetric, decentered or eccentric with respect to the visual axis.

13. The optical aberrations of claim 8 comprising one or more of defocus, prism, tilt, astigmatism, coma, trefoil and tetra foil.

14. The preferred retinal locations of claim 8 comprising non-foveal areas of retina located 0.01 to 3.0 mm from the fovea which show functional sensitivity on a preoperative microperimetry examination.

15. The treatment method of claim 8 comprising a temporary alteration whereby the cornea gradually returns to its original shape so that it can be retreated with a more eccentric pattern in order to reach remaining PRLs more distant from the fovea in a progressing macular disease.
